(19) United States
(12) Patent Application Publication
(10) Pub. No.: US 2013/0337001 A1
(43) Pub. Date: Dec. 19, 2013

(54) Immunogenic composition for treatment of hepatitis C, a method for preparing the composition and use thereof for treating hepatitis C

(71) Applicant: Mikhail Valentinovich Filatov, Saint Petersburg (RU)

(72) Inventor: Mikhail Valentinovich Filatov, Saint Petersburg (RU)

(21) Appl. No.: 13/686,425

(22) Filed: Nov. 27, 2012

Related U.S. Application Data
(63) Continuation-in-part of application No. PCT/RU2011/000380, filed on May 26, 2011.

(30) Foreign Application Priority Data
May 27, 2010 (RU) 2010121252

(51) Int. Cl. A61K 39/29; A61K 35/14

(52) U.S. Cl. CPC A61K 39/29; A61K 35/17
USPC 424/189.1; 530/350; 435/283.1

(57) ABSTRACT

This disclosure provides an immunogenic composition comprising an amount of isolated dendritic cells comprising at least one fragment of a hepatitis C protein selected from the group consisting of the core protein of hepatitis C and the NS3 protein of hepatitis C, the fragment does not suppress activity of dendritic cells and is effective to produce an immunogenic response against hepatitis C infection and optionally T-lymphocytes activated for a Th1 response. This disclosure also provides a method and a kit for preparing the above composition. Further, this disclosure provides a method for treating or reducing the severity of hepatitis C and a method for enhancing immunoresponse in a patient suffering from hepatitis C virus infection by administering to the patient in need of the treatment the above composition. Finally, this disclose provides a use of the above composition for treating hepatitis C virus infection.

### CROSS-REFERENCE TO RELATED APPLICATIONS

This application is a continuation-in-part of International Application No. PCT/RU2011/00380, filed May 26, 2011, which claims the benefit of Russian national patent application No. 2010121252, filed May 27, 2010, the entire content of each being incorporated herein by reference. International Application No. PCT/RU2011/000,380 was published in English as WO2011/149389.

### FIELD OF THE INVENTION

The present invention relates to medicine, immunology and can be used for preventing or treating hepatitis C and viral hepatitis C-related conditions, especially in such cases where standard treatments are not efficient.

### BACKGROUND OF THE INVENTION

Viral hepatitis C (HCV) caused by hepatitis C virus is a widely spread disease. May 19th is even declared the International Day for cure of hepatitis C. Around 170 million people worldwide are infected with hepatitis C, and in 80% of these cases, the infected develop the chronic infection, potentially leading to liver cirrhosis and hepatocellular carcinoma [1].

For example, hepatitis B and hepatitis C infections are two of the most serious problems in the Russian Federation healthcare. There thought to be several million carriers of these infections, however the number of people who get the appropriate treatment is only in the thousands (See, for example, http://medportal.ru/mednovosti/main/2010/05/19/hepday/).

At the present moment the generally accepted opinion is that there is no single efficient treatment of hepatitis C infection (chronic HCV infection). Nonetheless, the standard method of treatment of hepatitis C is the treatment with interferon and ribavirin. For example, one way of treating chronic viral hepatitis is to administer to a subject an anti-viral composition where the interferon is administered through a catheter in the round ligament of the liver at 3,000,000 U every other day during the course of two weeks, repeating the treatment 3 times with 3 months intervals [2].

The most efficient method of treatment of hepatitis C today is the combination pegylated-interferon with ribavirin, but patients undergoing this treatment experience a good outcome in only about 50% of cases.

Another known method of treatment of chronic hepatitis C is by using group B vitamins, hepatoprotectors, and cycloferon, characterized in that in addition to receiving cycloferon patients a treated with REAFERON intramuscularly at a dose of 3 million International Units (IE) 3 times a week for 18-20 days, and treatment with cycloferon is extended up to 7-9 weeks with cycloferon administered once in 5 days at a dose of 600 mg. Two to four courses of combination therapy with REAFERON and cycloferon are conducted [5]. Another method of treatment of chronic hepatitis includes administration of hepatoprotector liproksol in one capsule, 2 times a day, with a dose of 2 g twice a day for 21-28 days.

Among other disadvantages of these and other known preparative methods for treatment of hepatitis are their lacks of efficacy, especially against chronic hepatitis C.

A number of immunotherapy treatments are currently being developed, which can potentially be less toxic and more effective for treatment at different stages of the disease. For example, it is known that the immune system is able to distinguish between the “self” and the “foreign”, and under normal conditions, an immune response against foreign or “not self” antigens is occurring. Virus infected cells are recognized by the immune system as foreign. However, such a natural immune response is often not strong enough to block the development of viruses.

The purpose of immunotherapy is to enhance the ability of the immune system to recognize cells infected with viruses and to establish effective mechanisms to reduce viral load. One important issue for any immunotherapy is the selection of antigens for directional effects and optimal presentation of antigens to the immune system.

There is a method of treatment of chronic hepatitis or cirrhosis of the liver that includes the infusion to a patient's blood of the mononuclear cells of umbilical cord from a donor, having a set of antigens HLA-A, HLA-B, HLA-DR, that differs from a recipient's antigens HLA-A, HLA-B, HLA-DR by not less than four antigens. For this type of therapy, the number of input cells ranges from 2.5×10⁶ to 10×10⁶ cells per kilogram of body weight per day, and the rate of introduction of mononuclear cells from the umbilical cord blood donor is from 2 to 10 times with an interval of 10-60 days [7].

It is known, however, that, even if successful repopulation donor cells occurs, other transplanted cells still may cause a severe immunopathologic effect, and in particular, a “graft versus host” reaction is possible, even for donor-recipient pairs having an identical HLA type.

A vaccine composition for treating or preventing hepatitis infection, in particular, hepatitis B, is known. The vaccine consists of the antigen of hepatitis B virus and an acceptable carrier, such as aluminate, with 3-O-deacylated monophosphoryl-lipid A. A combination of vaccines that include the specified vaccine preparation and method of preventing hepatitis are also known [8].

Various vaccines exist which are preventive vaccines against hepatitis B [9, 10]. There are various preventive vaccines against hepatitis B virus and/or hepatitis C, including a nucleic acid molecule that contains a partial gene-fused hepatitis C virus genome and hepatitis B, or an incomplete genome of the hepatitis C virus, comprising the nucleotide sequence encoding the full core protein of hepatitis C and the S-protein gene of hepatitis B, or an incomplete virus genome of hepatitis C, comprising the nucleotide sequence encoding the full core protein of hepatitis C, connected to the regulatory elements functional in human cells, as well as other pharmaceutical compositions containing such structures [11].

However, the above-described vaccines and methods of vaccination may have a mostly prophylactic value associated with a possible reduction in viral spread after the immediate infection. Known vaccines are virtually ineffective therapeutic agents in the presence of a large viral load. A vaccine for the treatment of hepatitis C, especially in the chronic form, is not known.

Prerequisites for the development of vaccines that have therapeutic value became possible as a result of the development of anti-cancer vaccines for antitumor immunization, based on the dendritic cells that are loaded with antigenic material. Dendritic cells' use as immuno-stimulating agents is of growing interest for both in vivo and in vitro applications. For example, a method used in oncology for the treatment of tumors includes the administration of dendritic cells induced in vitro for the beginning of maturation and characterized by their ability to capture and process an antigen in vivo. Pharmaceutical compositions used in these methods include the dendritic cells in combination with a pharmaceutically acceptable carrier for their administration [12]. This method and pharmacological composition provide induction of antitumor immune response by efficiently capturing and processing, by dendritic cells, of tumor antigens at the site of a tumor, secretion of the cytokines and contact with T-cells in the lymph node. However, this is not effective in treating viral hepatitis, since it does not provide a reduction in viral load.

There is a method for prevention and treatment of cancer and immunodeficiencies stemming from viral and bacterial infections that includes obtaining of activated lymphocytes (LAK) and dendritic cells (DC) and comprising: a) isolating of mononuclear leukocytes (ML) from blood or bone marrow of the subjects; b) incubation of the isolated mononuclear lymphocytes in a culture medium; c) separation of MLs into adherent and not-adherent to the plastic culture flask, d) processing of adherent MLs to transform them into dendritic cells, e) treatment of DCs with tumor lysates; and f) The cultivation of not-adhering to plastic lymphocytes with interleukin-2 to generate LAK cells [13]. The method of treating and preventing cancers, infectious diseases and immunodeficiency states comprising combination of dendritic cells and LAK cells provides a combined effect on the innate and adaptive immunity, but is not sufficiently effective in treating hepatitis and other viral diseases.

There are methods of differentiation: of monocytic precursors of dendritic cells into immature dendritic cells that are well known in cancer treatments. For example, the production of mature dendritic cells using granulocyte-macrophage colony stimulating factor GM-CSF without other cytokines under conditions that prevent cell adhesion to the flask, providing contact of differentiated dendritic cells' precursors with an antigen of interest for a period of time sufficient to absorb the antigen. The antigen can be the tumor-specific antigen, tumor-associated antigen, viral antigen, bacterial antigen, tumor cells, nucleic acids encoding the antigen isolated from tumor cells, bacterial cells, recombinant cells expressing the antigen, cell lysates, membrane preparations, recombinant antigen, peptide antigen or isolated antigen [14].

There is an autologous vaccine for treating oncological diseases, such a vaccine that includes obtaining lymphocytes that have been activated with interleukin-2 and dendritic cells that have been obtained by incubating the immature dendritic cells with a tumor lysate. The vaccine has T-lymphocytes that were specifically activated by mature dendritic cells and is characterized by the manner in which it was produced. The method of preparation includes the isolation of mononuclear lymphocytes (MNK) from the peripheral blood of a subject, cultivation of MNKs in the DMEM medium, separation of cells into monocytes that adhere to the substrate and lymphocytes that do not adhere to the substrate, placing of the MNKs into a culture medium, isolation of non-adhering lymphocytes and subsequent addition of IL-2 to obtain lymphokin-activated killer cells (LAK-cells), adding to a remanding adhering monocytes of a growth factor, stimulating DC cells' maturation by an autologous tumor lysate in vitro and the subsequent addition of maturation factors for the duration of 1 day. For the isolation of adhering monocytes, MNKs are cultivated in the medium augmented with 10% FSC for 1 hour, after which immature dendritic cells are obtained. The adhering monocytes are cultivated in a medium with neupogen at a 50 ng/ml concentration during 48 hours, and after obtaining mature dendritic cells, the immature DCs are cultivated in a medium with 2000 MU/ml of Reoferon and 50 ng/ml beta-leukin. At the same time, LAK cells from the non-adhering fraction of MNK cells are cultivated in a medium containing 100 U/ml Roncoleukin for 72 hours. The mDCs and LAKs are washed by centrifugation in saline solution for 10 min at 1500 rpm, after which mDCs and LAKs are cultivated together in a medium with 100 U/ml of roncoleukin for 24 hours, after which the adhering fraction and non-adhering fraction are washed separately by centrifugation in saline solution for 10 min at 1,500 rpm. The resultant population comprises the vaccine [15].

Neither instance for treating viral infections including hepatitis C infection using dendritic cells, nor methods for treating hepatitis C infection using combinations of dendritic cells and activated T-lymphocytes were disclosed. Viral infections were not known to be treated with the cell vaccine characterized by switching off the genes SOCS1, CTLA4, FAS [symbol] FOXP3 in dendritic cells and/or T-lymphocytes by introducing of the corresponding siRNAs.

A method of treatment of malignant brain tumors was disclosed, comprising the isolation of a patient's blood monocytes, cultivation with growth factors, and addition of the monocytes to those dendritic cells obtained from monocytes of the antigenic material from patients' tumor. The resulting dendritic cells are injected back into the patient subcutaneously, in conjunction with additional immune modulation by activated lymphocytes [16]. This method purportedly allows activation of specific antitumor immunity and is characterized by the fact that the patients' peripheral blood mononuclear cells are obtained and cultivated with growth factors, then added to a medium with dendritic cells obtained from patient monocytes. The antigenic material prepared from a patient's tumor is added by electroporation, then the dendritic cells are injected intradermally, followed by immunomodulation using activation by phytohaemagglutinin lymphocytes of a patient. At this point, in addition to dendritic cells, autologous activated lymphocytes are injected, providing production of cytokines that can catalyze the immunological antitumor response.

With such a method, a fragment of a patient's tumor that has been taken during the surgery is dissociated into protein extract, to be used as the antigen in later work. The patient's peripheral blood monocytes are cultivated with growth factors. The antigenic material is added first to monocytes, and then to the dendritic cells. Next, dendritic cells are injected intradermally into a subject. In parallel, immunomodulation of the patient is performed with activated lymphocytes, wherein the lymphocytes were derived from mononuclear leukocytes of the subject, and activated by phytohaemagglutinin. Activated lymphocytes are then also injected intradermally.

Thus, adequately presenting a tumor antigen to immunocompetent cells in combination with a nonspecific immunity (due to activated lymphocytes) can activate specific antitumor immunity and enables the use of this method as an adjuvant treatment of complex antineoplastic treatments. In that way, the method provides treatment of malignant brain tumors, reducing side effects and complications, increases the life expectancy of patients, and adds to the quality of life of the patient by offering T-lymphocytes of tumor antigen “professional” antigen-presenting cells of the body, i.e., dendritic cells which contain the tumor antigen.

A disadvantage of the method, though, is its failure to treat hepatitis with their predominantly viral nature. Because the use of dendritic cells loaded with tumor antigens from the patient as the prototype, the method requires obtaining antigenic material of an individual tumor, which significantly complicates the application of this approach.

Developing a vaccine strategy against hepatitis B virus (HBV) and/or hepatitis C virus (HCV) is complicated not only by a significant heterogeneity of isolates of HBV and HCV, but also by the fact that a mixture of heterogeneous genomes exists within isolates [17].

Vaccination and immunization apply mainly to the introduction of non-virulent factors against which the immune system of the individual may initiate an immune response, which will be useful for protection against the pathogen. The immune system: identifies invading “alien” composition by the identification of proteins and other large molecules, which, by nature, are absent in the individual. Foreign protein is a target upon which the immune response is produced.

A prototypical method of therapy for treating a viral infection in a subject was disclosed in US patent application 2010/0143405. The method comprises harvesting cells from a subject; generating a concentrated population of dendritic cells from the harvested cells; exposing the dendritic cells to lipopeptides comprising T helper and viral killer-T cell (CTL) epitopes and/or antibody epitopes; and re-introducing the dendritic cells into the subject. The authors used lipopeptide compounds as antigens. The lipopeptide compounds consist of short (9-11 amino acids) peptides corresponding to putative immunogenic epitopes of viral proteins (Core, NS3 and NS4) having a lipid moiety attached via the terminal side chain epsilon-amino group of an internal lysine or lysine analog of the peptide. Such lipopeptide compounds were described earlier in WO04/014956 and WO04/014957. But unfortunately, results of clinical investigation was negative [30]. The conclusion of the authors was unfavorable: “Immunotherapy using autologous dendritic cells pulsed with lipopeptides was safe, but was unable to generate sustained responses or alter the outcome of the infection”.

NS3 protein is most conservative and immunogenic protein of all known subtypes of hepatitis C virus. NS3 protein contains immunodominant epitopes activating CD4 T helpers and CD8 cytotoxic lymphocytes detectable in the self-recovered patients [31].

Investigation of NS3 immunogenicity revealed its epitopes highly efficient in activation of CD8 cytotoxic lymphocytes. And the most immunogenic epitope is located in the N-terminus fragment of the protein exhibiting serine protease activity (amino acids 1038-1047) [32].

Use of short protein fragments as antigen material may be ineffective due to the significant variability generated in the virus replication process [33]. But use of full-length protein NS3 is not reasonable since its regulatory and enzymatic activities may influence on immunogenic properties of dendritic cells and lymphocytes. It was found that full-length proteins of hepatitis C virus including NS3 protein are able to significantly disturb functioning both immatured and matured dendritic cells [34].

N-terminus of hepatitis C NS3 protein exhibits serine protease activity and also interacts to several proteins of host cell as well. For example, binding of N-terminus of NS3 protein to ELKC proteins involved into system of intracellular transport and secretory pathways was demonstrated [35].

Structural and non-structural proteins of hepatitis C virus including NS3 protein are able to suppress interferon induction and block the antiviral mechanisms. Thus it was shown that NS3 protein blocks phosphorylation, dimerization and translocation of IRF3 protein (interferon regulatory factor) into nucleus resulting in activation of the protein [36]. And it was also shown that NS3 protein is able to induce apoptosis of dendritic cells [37]. Therefore, the establishment of effective drugs, vaccines and medical treatment is essential, and, despite notable achievements in this area remains problematic.

### BRIEF SUMMARY OF THE INVENTION

The objective of the present invention was creating a composition for treating or preventing hepatitis C virus infection.

The objective was achieved by creating a composition comprising the dendritic cells derived from monocytes from a subject's blood, fragments of a hepatitis C virus proteins, mainly the fragments of conservative proteins, e.g., a core protein or/and NS3 protein, and/or dendritic cells presenting the fragments of the hepatitis C protein NS3 and a pharmaceutically acceptable carrier and/or adjuvant. The authors of the present invention have realized that composition comprising the dendritic cells loaded with extensive fragments of the hepatitis C NS3 protein rather than short fragments of NS3 protein or full-size NS3 protein can significantly enhance immunoresponse in a patient suffering from hepatitis C virus infection.

In an embodiment, an immunogenic composition for treating or preventing hepatitis C infection comprises an effective amount of isolated dendritic cells loaded with at least one fragment of a hepatitis C protein or a variant thereof, wherein the protein is selected from the group consisting of the core protein of hepatitis C and the NS3 protein of hepatitis C, the fragment does not suppress activity of dendritic cells and/or is effective to produce an immunogenic response against hepatitis C infection and a pharmaceutically acceptable carrier and/or adjuvant and optionally T-lymphocytes activated for a Th1 response. In the certain embodiment, the fragment of a hepatitis C protein is introduced into the dendritic cells by electroporation. In another embodiment, the fragment of a hepatitis C protein comprises amino acids 1027-1218 of the NS3 protein or a variant thereof, wherein the said fragment or the variant thereof is characterized by at least one of the following: (a) does not suppress activity of dendritic cells, (b) effective to produce an immunogenic response against hepatitis C infection and (c) reduces the viral load in the patient. In another embodiment, the dendritic cells are developed from the monocytes from a subject's blood. In the certain embodiment, the dendritic cells are modified to suppress expression of at least one of the genes selected from the group consisting of SOCS-1 and FAS. In another embodiment, the dendritic cells are present at a quantity of 5×10⁵-5×10⁷ cells per dose of the composition according to the present invention. In another embodiment, the T-lymphocytes are isolated from a subject's blood. In the certain embodiment, the T-lymphocytes are modified to suppress expression of at least one gene selected from the group consisting of CTL4, FAS and FOXP3. In certain other embodiment, the T-lymphocytes are present at a quantity of 5×10⁶-5×10⁷ cells per dose of a composition according to the present invention. In the certain embodiment, the suppression of the expression of at least one of the genes selected from the group consisting of SOCS-1 and FAS in the dendritic cells and at least one of the genes selected from the group consisting of FAS, CTLA4, and FOXP3 in T-lymphocytes is performed by introducing short double-stranded interference RNA corresponding to the at least one gene selected from the group consisting of SOCS-1 and FAS in the dendritic cells, and FAS, CTLA4, and FOXP3 into the T-lymphocytes, respectively.

In another embodiment, a fragment of a hepatitis C protein which does not suppress activity of dendritic cells and is effective to produce an immunogenic response against hepatitis C infection comprises amino acids 1027-1218 of the NS3 protein or a variant thereof.

In another embodiment, a method for preparing the composition of claim 1 comprises:
(a) preparing dendritic cells comprising a fragment of a hepatitis C protein by: providing an amount of isolated autologous dendritic cells;
   loading the said dendritic cells with at least one fragment of a hepatitis C protein or a variant thereof, wherein the said fragment or the variant comprises amino acids 1027-1218 of the NS3 protein, does not suppress activity of dendritic cells, effective to produce an immunogenic response against hepatitis C infection; and
   combining said dendritic cells with a pharmaceutically acceptable carrier and/or adjuvant; and optionally
(b) isolating the T-lymphocytes from a subject's blood and its activating to produce a
   Th-1 type response,
(c) combining the dendritic cells with T-lymphocytes which have been activated to produce a Th-1 type response in the composition.

In another embodiment, the fragment of NS3 protein is introduced into the dendritic cells by electroporation. In another embodiment, the fragment of a hepatitis C protein comprises amino acids 1027-1218 of the NS3 protein or a variant thereof, wherein the said fragment or the variant thereof is characterized by at least one of the following: (a) does not suppress activity of dendritic cells, (b) effective to produce an immunogenic response against hepatitis C infection and (c) reduces the viral load in the patient. In certain embodiment, the dendritic cells are further modified to suppress expression of at least one gene selected from the group consisting of CTL4, FAS and FOXP3, and the T-lymphocytes are further modified to suppress expression of at least one gene selected from the group consisting of CTLA4, FAS and FOXP3. In certain other embodiment, the suppression of the expression of at least one of the genes selected from the group consisting of SOCS-1 and FAS in the dendritic cells and at least one of the genes selected from the group consisting of FAS, CTLA4, and FOXP3 in T-lymphocytes is performed by introducing short double-stranded interference RNA corresponding to the at least one gene selected from the group consisting of SOCS-1 and FAS in the dendritic cells, and FAS, CTLA4, and FOXP3 into the T-lymphocytes respectively.

In another embodiment, a method for treating, preventing or reducing the severity of hepatitis C virus infection in a patient in need thereof is provided that comprises administering to the patient an effective amount of a composition according to the present invention as described above. In another embodiment, the method further comprises the use of traditional methods of treatment of a hepatitis C virus infection.

In another embodiment, a method for enhancing immunoresponse in a patient suffering from hepatitis C virus infection comprises administering to the patient in need of the treatment the composition described above.

In another embodiment, use of a composition described above for treating hepatitis C virus infection comprises administering said composition to a patient in need thereof.

Yet in another embodiment the invention provides a unit dosage form for the treatment or prophylaxis of hepatitis C virus infection in a patient in need thereof, comprising an effective amount of the composition according to the present invention.

In another embodiment, a system or kit for preparing the composition described above comprises at least one fragment of a hepatitis C protein selected from the group consisting of the core protein of hepatitis C and the NS3 protein of hepatitis C, the fragment does not suppress activity of dendritic cells and is effective to produce an immunogenic response against hepatitis C infection, and means for loading the fragment into the dendritic cells isolated form the patient to be treated with said fragment of the hepatitis C protein or variant thereof, optionally, by electroporation. In certain embodiment, the fragment of a hepatitis C protein comprises amino acids 1027-1218 of the NS3 protein or a variant thereof.

### BRIEF DESCRIPTION OF THE FIGURES

FIG. 1 shows diagram illustrating method of measuring the rate of cell population passed through the cell cycle.

FIG. 2 shows diagram of stimulation of T-lymphocytes proliferation by phytohaemagglutinin.

FIG. 3 shows diagram of stimulation of T-lymphocytes proliferation by phytohaemagglutinin after trasnfection with mixture of small interfering RNAs (siRNA) specific for genes of interest.

FIG. 4 shows diagram of stimulation of T-lymphocytes proliferation by phytohaemagglutinin after trasnfection with mixture of small interfering RNAs (siRNA) non-specific for genes of interest.

### DETAILED DESCRIPTION OF THE INVENTION

In an embodiment, it is an objective to develop a new immunogenic composition for treating or preventing hepatitis C infection, e.g., a cell-based vaccine, and to develop a method of treatment or prevention for hepatitis C in chronic form, particularly in cases where treatment with a standard combination of interferon and ribavirin is not effective, with a measurable result of a decrease in the concentration of virus in the blood.

As used herein, the following terms have the following meanings:

“Viral NS3 proteins” generally refers to the NS3 protein of hepatitis C virus, with a molecular weight of 70 kDa. It contains two different domains with separate functions. The N-terminal portion is a serine protease responsible for processing the C-terminal part of the polyprotein predecessor. The C-terminal portion of NS3 functions as the RNA helicase. It is believed that the NS3-helicase untwists viral RNA in the early stages of genome replication.

“Core protein” refers to a structural viral capsid protein C (core protein). Capsid protein of HCV, detectable in the serum of infected patients, the molecular weight corresponds to p21. Amino acid sequence of protein C in all six genotypes of HCV highly conserved. The protein can be divided into three domains. Antibodies to this protein detected in the process of natural infection with hepatitis C.

“Fragment of a hepatitis C protein” means fragment of a protein presented in hepatitis C virus particles. The fragment according to the present invention is preferably selected from the group consisting of the core protein of hepatitis C and the NS3 protein of hepatitis C, preferably is a fragment of NS3 protein. The fragment according to the present invention does not suppress activity of dendritic cells and is effective to produce an immunogenic response against hepatitis C infection.

Phrase “a fragment of hepatitis C virus protein does not suppress activity of dendritic cells” means that the fragment does not change functioning, characteristics and survival of dendritic cells. Preferably, a fragment of the present invention is a fragment of hepatitis C virus protein, which has not any activity suppressing dendritic cells or T lymphocytes functions. Whether the fragment does not suppress activity of dendritic cells can be easily determined as described in the articles Jirmo A C et al and Krishnadas D K et al [34] incorporated herein by reference.

“Immunogenic,” as used herein, refers to the ability to elicit an immune response (e.g., cellular or humoral) in a patient. In particular, antigens that are immunogenic (and immunogenic portions or other variants of such antigens) are recognized by a B-cell and/or a T-cell surface antigen receptor. Antigens that are immunogenic (and immunogenic portions or other variants of such antigens) are capable of stimulating cell proliferation, interleukin-12 production and/or interferon-γ production in one or more cells selected from the group of T cells, NK cells, B cells and macrophages, where the cells are derived from the patient.

The particular example of the fragment of hepatitis C virus protein is fragment of NS3 protein of hepatitis C virus comprising amino acids corresponding to amino acids 1027-1218 of the NS3 protein or a variant thereof. Such fragment is represented by amino acids 20-213 of SEQ ID NO: 2 or a variant thereof.

Substitution variants include those fragments wherein one or more amino acid residues in an amino acid sequence are removed and replaced with alternative residues. In some embodiments, the substitutions are conservative; however, this disclosure embraces substitutions that are also non-conservative. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table I (see, for example, WO 97/09433, page 10, published Mar. 13, 1997), immediately below. Amino acids on the same block of second column and preferably in the same line of the third column may be substituted for each other.

TABLE 1
Conservative Substitutions I

| Side chain | Characteristic | Amino acid |
|---|---|---|
| Aliphatic | Non-polar | G, A, P |
| | | I, L, V |
| | Polar, uncharged | S, T, M |
| | | N, Q |
| | Polar, charged | D, E |
| | | K, R |
| Aromatic | | H, F, W, Y |
| Other | | N, Q, D, E |

Alternatively, conservative amino acids can be grouped as described in Lehninger (Biochemistry, Second Edition; Worth Publishers, Inc. NY: N.Y. (1975), pp. 71-77) as set out in Table 2, immediately below.

TABLE 2
Conservative Substitutions II

| Side chain | Characteristic | Amino acid |
|---|---|---|
| Non-polar (hydrophobic) | Aliphatic | A, L, I, V, P |
| | Aromatic | F, W |
| | Sulfur-containing | M |
| | Borderline | G |
| Uncharged (polar) | Hydroxyl-containing | S, T, Y |
| | Amides | N, Q |
| | Sulfhydryl | C |
| | Borderline | G |
| Positively charged (basic) | | K, R, H |
| Negatively charged (acidic) | | D, E |

Variants or derivatives can also have additional amino acid residues which arise from use of specific expression systems. Variants which result from expression in some vector systems are also contemplated, including those wherein histidine tags are incorporated into the amino acid sequence, generally at the carboxy and/or amino terminus of the sequence.

Deletion variants are also contemplated wherein one or more amino acid residues in a binding domain of this disclosure are removed. Deletions can be effected at one or both termini of the protein, or from removal of one or more residues within the amino acid sequence.

The term “variants” also includes a set of NS3 protein fragments shorter than 1027-1218 aa fragment of NS3 protein, alignment of which can form 1027-1218 aa fragment of NS3 protein of the present invention or conservative variants thereof, allowing dendritic cells to expose generally the same set of epitopes as 1027-1218 aa fragment of NS3 protein or conservative variants thereof. The 1027-1218 amino acids fragment of NS3 protein according to the present invention can be obtained by, for example, expressing a DNA fragment encoding the fragment in suitable host cell, for example in E. coli cells as it is described in the Example 1. In an embodiment, 1027-1218 amino acids fragment of NS3 protein is bound to the N-terminal amino acids using a polyHis tag suitable for affinity purification plus site of thrombin protease. Alternatively, the fragment can be obtained by chemical synthesis.

Methods for preparation of plasmid DNA, digestion and ligation of DNA, transformation, expression, selection of an oligonucleotide as a primer, and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

“Dendritic cells” (DCs) refers to a heterogeneous population of antigen-presenting cells of the bone marrow origin. Morphologically, the dendritic cells are large cells (15-20 microns) round, of oval or polygonal shape, with off-center located nucleus, numerous branched processes of the membrane. Dendritic cells express a set of surface molecules characteristic of other antigen presenting cells: receptors for cell wall components and nucleic acids of microorganisms, including the receptors for complement components and toll-like receptors, the molecules of class II major histocompatibility complex (MHC); costimulatory molecules CD40, B7½ (CD80, CD86), B7-DC, B7-H1; intercellular adhesion molecules (ICAM-1). Dendritic cells can be produced easily from peripheral blood monocytes and can effectively present the antigen of T-lymphocytes. To date, many studies on the modulation of immune response in patients with chronic infectious diseases and cancer using dendritic cells primed with antigen were done.

“Antigen” refers to substances that cause specific to them cellular or humoral immune response.

“Monocytes” refers to a large group of mature single-core agranulocytic leukocytes 12-20 microns in diameter with an eccentrically located polymorphic nucleus with loose chromatin network, and azurophilic granules in the cytoplasm. Monocytes have an unsegmented nucleus, and are the most active phagocytes in peripheral blood. The cells have an oval shape with a large bean-shaped, chromatin-rich nucleus and a large cytoplasm having many lysosomes. Normally, monocytes comprise from 3% to 11% of the total number of blood leukocytes. In an embodiment, there are approximately 450 monocytes in 1 μl of blood. Monocytes are resident in the blood for 2-3 days, after which they go into the surrounding tissues, where, having reached maturity, they become tissue macrophages or dendritic cells.

“T-lymphocytes” or “T-cells” refer to lymphocytes, the developing in the thymus of mammals from precursors, pre-thymocytes, derived from the red bone marrow. In the thymus T-lymphocytes differentiate, acquiring T-cell receptor (TCR) and surface markers. They play an important role in adaptive, that is, acquired immune response. Provide recognition and destruction of cells bearing foreign antigen, enhances the action of monocytes, NK-cells, as well as take part in the switching of immunoglobulin isotypes from early immunoglobulin IgM, the later production of IgG, IgE, IgA in B-cells.

“Activated T-lymphocytes” refers to T-lymphocytes exposed to inflammatory cytokines and agents that stimulate their proliferation (phytohaemagglutinin) for the implementation of Th1 response as well as T-lymphocytes obtained as above where the expression of genes CTLA4 and/or FAS and/or FOXP3 has been suppressed.

“Composition for treatment or prophylaxis/prevention of hepatitis C” generally refers to a therapeutic or prophylactic cell vaccine designed to treat hepatitis C, prepared on the basis of the dendritic cells loaded with antigenic (immunogenic) material and, optionally, phytohaemagglutinin-activated T lymphocytes, used directly as such or in an amplified version—in conjunction with the temporary suppression of the genes SOCS1, FAS, CTLA4, and FOXP3 by introducing siRNA into cells.

Action of cell vaccines comprising dendritic cells and activated T-lymphocytes is not always predictable. Thus, compositions comprising dendritic cells presenting tumor antigens sometimes causes immunosuppression instead of antigen-specific immune response. Use of recent technique allowing temporary suppression of a target gene expression by introducing short double-stranded interfering RNA may be very helpful [38]. Such technique allows to overcome immunosuppression condition of cells of immune system by temporary suppression of expression of genes suppressing proliferative activity of T-lymphocytes, particularly to overcome immunosuppressive properties of T-lymphocytes preventing its activation. Suppression of expression of FAS, CTLA4, SOCS1 and FOSP3 genes may be used for such purposes.

“A pharmaceutically acceptable carrier and/or adjuvant” encompasses any of such substances that are traditionally used in the art and, which do not have any substantially unfavorable influence on the effectiveness of the composition for treatment of hepatitis C.

The phrase “cells are modified to suppress expression of a gene” means that cells have been modified in such a way that the modified cells are unable to synthesize a product of the gene. Temporary suppression of genes is preferable in the present invention. Such temporary suppression of expression can be performed by, for example, using “small interfering RNA” (siRNA).

“Small interfering RNA” (siRNA) refers to short double-stranded RNA molecules capable of RNA interference.

“RNA interference” (also referred to as born RNA interference, RNAi) refers to small RNA molecules mediating the suppression of gene expression at the stage of transcription, translation or degradation of mRNA, Small interfering RNA (small interfering RNA, siRNA), representing a short double-stranded RNA molecules that bind to specific sequences of mRNA (usually in the coding region), leading to degradation of mRNA. Selective effect of RNA interference on gene expression makes RNAi a useful tool for studies using cell cultures and in living organisms, as synthetic double-stranded RNA introduced into cells, causing suppression of specific genes. For example, RNA interference is used to systematically “turn off” genes in the cells. The level of gene expression can be estimated by measuring the amount of protein translated from mRNA under consideration using Western blot.

“Gene expression” refers to a process in which genetic information from genes (DNA nucleotide sequence) is transformed into a functional product (RNA or protein).

“Immunosuppression” refers to suppression of immunity of an organism.

“SOCS1” refers to the gene coding a protein called a suppressor of cytokine signaling 1. This protein is a negative regulator of activation of macrophages and plays an important role in regulating autoimmune reactions involving dendritic cells. The suppression of SOCS1 gene expression breaks the tolerance of the immune system to its own antigens and may enhance the immune response.

“FAS gene” refers to a gene which encodes a receptor on the cell surface (FasR), which upon activation, causes programmed cell death. The emergence of FasR on dendritic cells and activated lymphocytes limits their viability.

“CTLA4” refers to a gene that encodes Cytotoxic T-Lymphocyte Antigen 4—a protein of immunoglobulin family whose appearance on the surface of activated T-lymphocytes leads to a suppression of cellular proliferation.

“FOXP3” refers to forkhead box P3, a gene that encodes the coding transcriptional regulator of T-lymphocytes that is necessary for the formation and functioning of regulatory cells that negatively regulate immune response.

A system or kit for preparing the composition of the present invention comprises at least one fragment of a hepatitis C protein selected from the group consisting of the core protein of hepatitis C and the NS3 protein of hepatitis C or variants thereof according to the present invention. The system or kit also comprises means for introducing the fragment into the dendritic cells obtained from the patient, optionally, by electroporation. Means may be compounds, reagents, containers and/or equipment. For example, one container within a kit may contain a fragment of a hepatitis C protein according to the present invention. One or more additional containers may enclose elements, such as reagents or buffers, to be used for preparing composition according to the present invention.

There is compelling evidence that the outcome of hepatitis C virus infection is determined by the specific immune response induced CD8 and CD4 T-lymphocytes [18]. Chronic hepatitis C infection is characterized by the absence of effective antiviral T-cell immune response. It is assumed that vaccination causes T-cell response against hepatitis C may be a strong preventive and therapeutic tool [19], but so far it has not been realized in practice.

The development of chronic infection is typically associated with lack of lytic activity of T lymphocytes and reduced ability to secrete interferon-gamma (IFNγ) [20]. Dendritic cells are the most effective antigen-presenting cells, so the immunization of these cells loaded with viral antigens in accordance with the present invention provides a new effective approach to therapy of hepatitis C.

Dendritic cells are a heterogeneous cell population with characteristic morphology and a widespread distribution in tissues, including blood. The cell surface of dendritic cells is unusual, with characteristic veil-like buds. Mature dendritic cells are usually identified as CD3−, CD11c+, CD19−, CD83+, CD86+ and HLA-DR+. Dendritic cells process and present antigens and stimulate the activation of T-cells and T-cell memory. Dendritic cells have a high ability to effectively present antigens to T-cells as major histocompatibility complex (MHC), and contribute to the initiation of the immune response by releasing cytokines that stimulating the activity of lymphocytes and macrophages.

Cells producing interferon-gamma play a key role in the control and eradication of hepatitis C. In particular, T cells specific for NS3 protein are associated with control of viremia [21]. Conserved proteins of the hepatitis C virus, which include the core protein and protein NS3, may represent a target for immunological control of infection and serve as a basis for new antiviral vaccines. In particular, in contrast to the envelope glycoprotein, which includes the hypervariable region in amino-terminal region of E2 [22], the core protein is highly conserved among different genotypes of HCV and generates an immune response of the host [23]. Studies show that most HCV infected individuals produce antibodies to core protein in early HCV infection [24].

However, as disclosed herein, an especially promising antigen for the induction of antiviral immunity is endogenous viral protein NS3. Patients whose immune system is able to control the spread of hepatitis C virus demonstrate the most powerful and sustained T-cell immune response to epitopes of viral protein NS3 [25]. NS3 contains a number of epitopes efficiently presented by HLA type-1 (HLA1) [26]. For example, patients who responds to therapy with interferon-alpha exhibit strong cytotoxic T cell activity specific to different epitopes of NS3 [27]. Furthermore, many epitopes known to be specific for hepatitis C virus are localized on the protein NS3 [28].

In an embodiment, a method disclosed herein includes a composition to be used complementary to conventional treatment, or as an individual application of a therapeutic or prophylactic cellular vaccine. In an aspect, a composition comprises activated T lymphocytes and dendritic cells of the patient to be treated, bearing as an antigen, a fragment of the viral protein NS3, or a mixture of fragments of the NS3 protein and the core protein, which allows an unexpectedly drastic reduction of the viral load in the patient, even with patients resistant to standard therapy.

In an embodiment, the compositions disclosed herein can be used by introducing into cells short interfering RNAs that temporarily switch off genes, suppressing the immune response: SOCS1, FAS, CTLA4 and FOXP3. As described herein, enhanced efficacy of the disclosed methods and compositions (therapeutic vaccines based on dendritic cells and/or activated lymphocytes) may be obtained by employing siRNA techniques to obtain a temporary shutdown of certain genes that are limiting the development of the immune response in the patient. The use of siRNA is described herein for use to affect one or more of a set of genes whose temporary shutdown would give the optimal improvement of efficacy of therapeutic vaccines used for treatment of hepatitis C.

In an embodiment, a immunogenic composition for treating hepatitis C infection (e.g. therapeutic cell vaccine) is prepared and used as follows:

Before each round of treatment for hepatitis C, 40-60 ml of peripheral blood of a patient is extracted into a syringe containing heparin at 50 U/ml. Blood must be received for processing no later than 36 hours after collection. Using the known methods, monocytes are obtained from blood and are cultivated at 37° C. in RPMI1640 medium supplemented with 10% human serum obtained from donors with a 4-th group of blood. Isolation of monocytes and lymphocytes is done by standard centrifugation on a stepwise gradient ficol-urografin (density solution 1.077 g/ml), followed by adhesion of monocytes to the surface of culture flasks.

For induction of formation of dendritic cells to the culture medium, growth factors are added as follows: 1) granulocyte-macrophage colony-stimulating factor in the concentration of 3000 U/mi, (it is possible to use pharmacological agents “Leukomax” by Novartis, or “Sargramostim” by Genzyme) and 2), interleukin 4 (IL4) in a concentration of 500 U/ml, which can be replaced by interleukin 15 or interferon alpha.

On the second day after the addition of growth factors to the medium, a mixture of RNA homologous to the genes SOCS1 and FAS is added, as well as an agent promoting its penetration into the cells—Lipofectamin (Dharmacon)®, prepared in accordance with the manufacturer's protocol. The remainder of the procedure for making of dendritic cells may be any method known in the art.

At 4-5 days, antigenic material is added to dendritic cells, mainly in the form: of fragments of viral protein NS3 and/or core protein. On the same day, to further strengthen the capture of antigenic material by dendritic cells, antigenic material is injected into the dendritic cells by electric charge (electroporation).

For the induction of full maturation of dendritic cells after the addition of antigen, proinflammatory signals are added: a conditioned medium derived from autologous mononuclear cultivation, or trace amounts of bacterial lipopolysaccharide (0.2 microgram per ml), or a mixture of cytokines TNFa+IL1b.

As a proof of obtaining true dendritic cells as a result of the above method, the following criteria may be used:

a) presence of growth in non-adhering to the substrate state (in contrast to macrophages, which are tightly adhered to the substrate);

b) a characteristic morphology of dendritic cells presenting multiple dendrites;

c) the emergence of a large number of surface markers characteristic of dendritic cells (HLA-DR, HLA-ABC, CD80, CD83) is determined using a fluorescent microscope or flow cytometry technique.

In parallel with the preparation of dendritic cells, the activated T-lymphocytes are optionally prepared from the same portion of blood, for which the mononuclear leukocytes are isolated by centrifugation in a density gradient by a standard procedure, and T-lymphocytes from the cell mixture are activated to proliferate by adding phytohaemagglutinin (20 micrograms 1 ml). The procedure is carried out as standard blast transformation of T-lymphocytes, except that for the primary stimulation of Th1-cell immune response, the bacterial lipopolysaccharide (0.2 micrograms per ml) is added to the cells.

In an embodiment, the siRNA specific for the genes FAS, CTLA4 and FOXP3 is injected into cells by electroporation prior to before adding phytohaemagglutinin.

The remainder of the procedure of preparing the T-lymphocytes is conducted in any manner known in the art.

On the sixth day, the dendritic cells loaded with antigen and T-lymphocytes activated to implement the Th1 response are combined together in 1.5 ml of medium in which they are cultured, and injected into the patient mainly paravertebrally in the interscapular region, intradermally, in 2 or 3 points on the back.

The preferential treatment of chronic hepatitis lasts for 5 weeks with weekly blood sampling and preparation of portions of dendritic cells loaded with antigen, mainly gene-engineered fragments of protein NS3 and/or core protein. Parallel to immunotherapy, a standard treatment of hepatitis, such as the one using interferon and ribavirin, may be employed. Upon completion of the treatment, the standard real time reverse transcription polymerase chain reaction (RT-PCR) is used to estimate the virus titer in the blood. If necessary, the treatment can be repeated.

### EXPERIMENTAL EXAMPLES

### Example 1

### Preparation of Recombinant DNA

The fragment of hepatitis C virus NS3 was made by cloning the synthetic E. coli codon-optimized DNA fragment coding the polypeptide of 192 amino acids (amino acids 1027-1218 from HCV protein NS3, where cloning was done at the sites of NdeI-XhoI of commercially available prokaryotic expression vector pET28a(+) (Novagen).

An expression vector compatible with bacterial (E. coli) strain BL21 (DE3) was prepared as in the pET Manual.

Induction of the expression is achieved by adding IPTG to the E. coli culture medium. The expressed recombinant protein contained from the N-terminus is a 20 amino acid fragment (polyHis-Tag for affinity purification plus the site for the protease thrombin, allowing isolation of the protein in the “pure” form) linked to a 192 amino acid fragment of NS3 HCV protein (amino acids 1027-1218).

Nucleotide sequence encoding such recombinant protein and amino acid sequence of the recombinant protein are disclosed in SEQ ID NO: 1 and 2, respectively.

The vaccine according to the present invention was prepared in the form of a composition comprising the following main ingredients: a suspension of autologous dendritic cells that were derived from the monocytes peripheral blood of the patient (0.5-1.0×10⁷ depending on the particular patient to be treated); a suspension of phytohaemagglutinin-activated autologous lymphocytes (3-5×10⁷ cells); 1 ml of saline; the fragment of HCV protein.

As it was shown in the following Examples, the positive results were obtained using the disclosed composition when the composition is used as a complex of dendritic cells in combination of almost universal viral antigens in the form of immunogenic fragments, mostly of NS3 protein or/and core protein, and T-lymphocytes activated for Th1 response, and having undergone the temporary suppression of the expression of genes SOCS1, FAS, CTLA4, and/or FOXP3.

### Example 2

Patient K having high titre of hepatitis C virus in blood (1b virus type, 10⁶-10⁷ virus particles/ml) during 3 years continued attempts to use standard therapy based on pegylated interferon-α and ribavirin in different specialized clinics. However, after such therapy titre of hepatitis C virus still remained high. Appearance of liver enzyme in blood was observed indicting liver fibrosis. Further, the increase of α-fetoprotein concentration in blood was observed along with the appearance of two loci of hepatoma malignant transformation. These loci were suppressed by laser surgery followed by another course of standard therapy based on pegylated interferon-α and ribavirin in combination with the therapy according to the present invention was applied. The patient was treated with 1.5 ml of composition comprising 10⁶ autologous dendritic cells comprising fragment of hepatitis C NS3 protein according to the present invention (10 μg of the fragment) and 10⁷ activated lymphocytes.

After the first course of therapy consisted of 5 rounds, the virus titre was decreased to 400 ME/ml, the level closed to detection level of standard PCR. During more than 2 years of continuous observation, the virus titre remains on the same detection level. No relapses of malignant transformation were observed.

### Example 3

Patient M. having high hepatitis C virus titre in blood (3a virus type, over 1000000 ME/ml) and associated with it liver lesion (alanine transaminase 150 U/l, asparagine transaminase 100 U/l) was treated with standard therapy based on interferon-α and ribavirin (pegasys 180 μg/week, ribavirin—1000 mg/day) during one year. As a result of the treatment decrease of liver enzymes level in blood was observed. Virus titre was decreased to the minimal level. However when the treatment was finished virus titre increased rapidly up to 380000 ME/ml. The patient was treated according to the therapy of the present invention. 1.5 ml of composition according to the present invention comprising 10⁶ autologous dendritic cells comprising fragment of NC3 protein of hepatitis C virus (10 μg of the fragment), and 10⁷ activated lymphocytes were injected to the patient every week during 3 months. Use of the therapy according to the present invention led to decrease of virus titre below the detection level (less than 750 ME/ml) and decrease of the liver enzymes concentration in blood (alanine transaminase 54 U/l, asparagine transaminase 43 UI). Observation during one year did not reveal any increase of virus titre.

### Example 4

Patient R having high hepatitis C virus titre (1b virus type, over 10⁶ ME/ml) was treated with standard therapy based on interferon-α and ribavirin (pegasys 180 μg/week, ribavirin 1200 mg/day) during half year. The therapy was unsuccessful, virus titer was not decreased significantly (the last data—5 800 000 ME/ml). Use of the therapy according to the present invention without injection of T-lymphocytes during 3 months led to significant decrease of virus titre (6500 ME/ml). 1.5 ml of composition comprising 10⁶ autologous dendritic cells comprising fragment of hepatitis C virus NS3 protein according to the present invention (10 μg of the fragment) without the activated lymphocytes and 50 pmol of anti-SOCS1 and anti-FAS siRNA was injected to the patient every week.

### Example 5

Patient B having high hepatitis C virus titre (1 050 000 ME/ml), remained during continuous time in spite of standard therapy was treated according to the therapy of the present invention. 1.5 ml of composition comprising 10⁶ autologous dendritic cells comprising fragment of hepatitis C virus NS3 protein (10 μg of the fragment) and 10⁷ activated lymphocytes were injected into the patient every week. After 3 months of the therapy virus titre was decreased to 2000 ME/ml.

### Example 6

### Temporary Suppression of Genes Reducing Immunogenity of Cells of the Composition According to the Present Invention

For evaluation of immunologous status of mononuclear cells, the so called reaction of T-lymphocytes blast-transformation was used. Ability of T-lymphocytes from peripheral blood to proliferate in response to phytohaemagglutinin action was estimated. For that purpose mononuclear lymphocytes isolated from peripheral blood was incubated during 4 days at 37° C. in the culture medium RPMI-1640 supplied with 10% of human serum and 10 μg/ml of phytohaemagglutinin. It is known that under such conditions T-lymphocytes are involved into cell cycle. But in case of immunosuppression, the amount of lymphocytes able to proliferate is decreased. Thus, measuring the percentage of lymphocytes activated by phytohaemagglutinin allows to evaluate the potential ability of immune system to respond by the T lymphocyte proliferation on an antigen stimulation. The percentage of cells divided during the incubation time was measured by flow cytometry. Quenching the Hoechst33258 dye after binding to cell DNA comprising bromodeoxyuridine incorporated into the DNA during replication was measured [39].

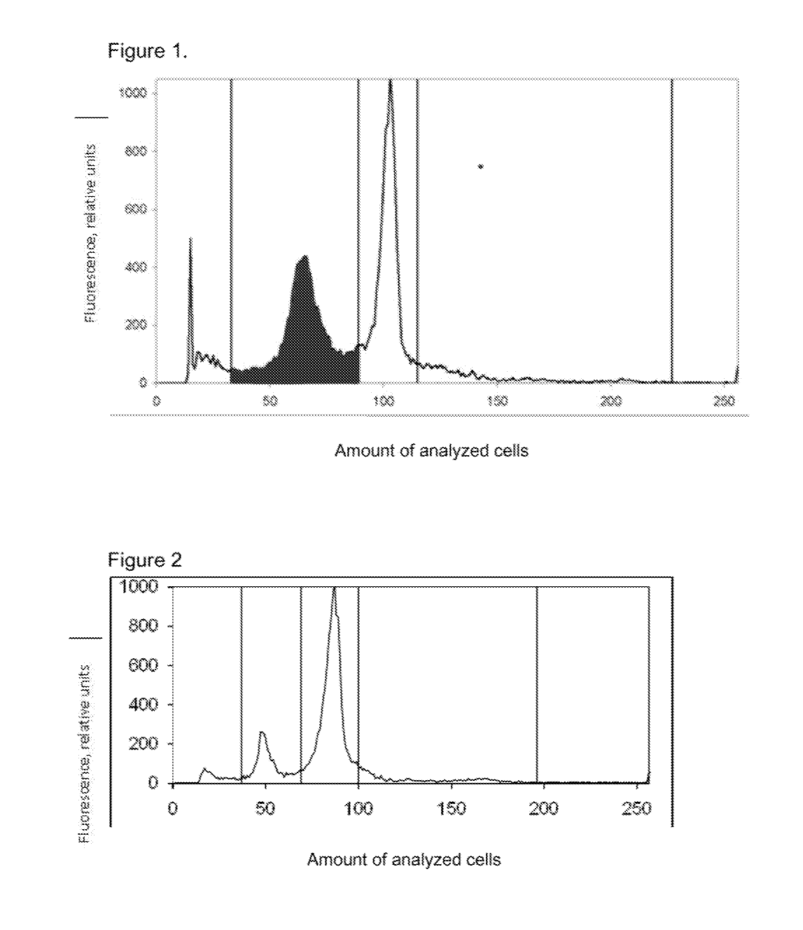

Fluorescence of cells comprising bromodeoxyuridine incorporated into DNA during replication was significantly lower than that of non-proliferated cells. Such proliferated cells are represented by left peak on the cytometry histogram, while non-proliferated cells are represented by right peak (FIG. 1). Area of the left peak allows estimating the percentage of cells proliferated during incubation with bromodeoxyuridine.

Percentage of cells able to proliferate in response to phytohaemagglutinin action significantly varies among different donors (30 to 40%) (FIG. 2). However, percentage of such cells in the patents suffering from some diseases may be significantly low (up to 0% in the immunosuppression condition).

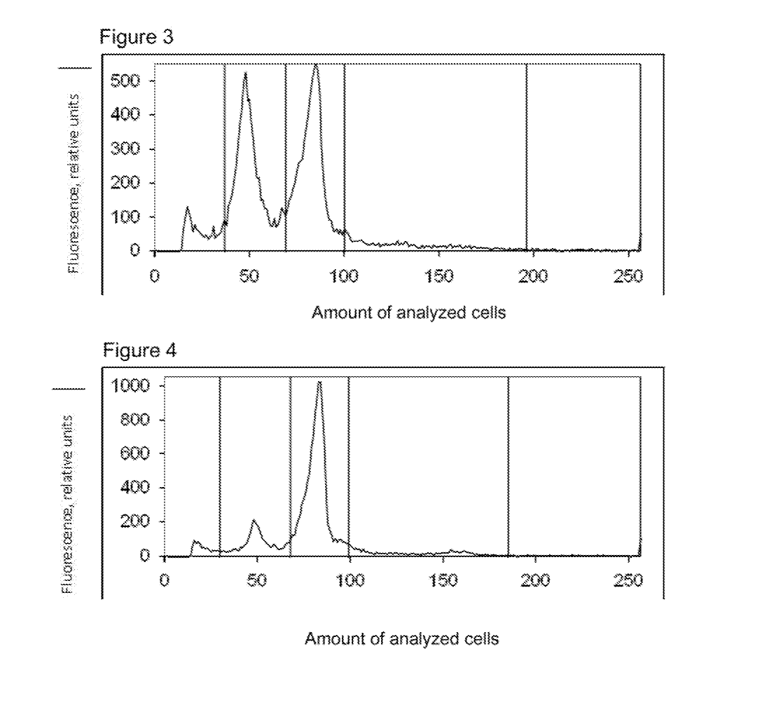

It was shown that temporary suppression of CTLA4, FAS, FOXP3 and SOCS1 genes expression significantly increases the percentage of the T-lymphocytes able to proliferate (FIG. 3). Transfection of cells with non-specific small double-stranded RNA did not change the percentage of the T-lymphocytes able to proliferate (FIG. 4).

Those of ordinary skill in the art will readily appreciate that the foregoing represents merely certain preferred embodiments of the invention, Various changes and modifications to the procedures and compositions described above can be made without departing from the spirit or scope of the present invention, as set forth in the following claims.

1. A therapeutic immunogenic composition for treating or preventing hepatitis C infection in a human patient, comprising:
an effective amount of isolated autologous dendritic cells loaded with
at least one fragment of a hepatitis C protein, or a variant thereof, wherein the fragment of a hepatitis C protein comprises amino acids 1027-1218 of the NS3 protein, does not suppress activity of dendritic cells and is effective to produce an immunogenic response against hepatitis C infection; and
a pharmaceutically acceptable carrier and/or adjuvant.

2. The composition of claim 1, wherein the fragment of a hepatitis C protein is loaded into the dendritic cells by electroporation.

3. The composition of claim 1, wherein the dendritic cells are developed from the monocytes of the patient to be treated.

4. The composition of claim 1, wherein the dendritic cells are modified to suppress expression of at least one of the genes selected from the group consisting of SOCS-1 and FAS.

5. The composition of claim 1, wherein a dose of the composition for the treatment of a human patient comprises from 5×10⁵ to 5×10⁷ of the said dendritic cells.

6. The composition of claim 1 further comprising T-lymphocytes activated for a Th1 response.

7. The composition of claim 6, wherein the T-lymphocytes are isolated from the blood of the patient to be treated.

8. The composition of claim 6, wherein the T-lymphocytes are modified to suppress expression of at least one gene selected from the group consisting of CTL4, FAS and FOXP3.

9. The composition of claim 6, wherein a dose of the composition for the treatment of a human patient comprises from 5×10⁶ to 5×10⁷ of the said activated T-lymphocytes

10. The composition of claim 4, wherein the suppression of the expression of at least one of the genes selected from the group consisting of SOCS-1 and FAS in the dendritic cells is performed by introducing into the dendritic cells short double-stranded interference RNA corresponding to the at least one gene selected from the group consisting of SOCS-1 and FAS.

11. The composition of claim 8, wherein the suppression of the expression of at least one of the genes selected from the group consisting of FAS, CTLA4, and FOXP3 in T-lymphocytes is performed by introducing short double-stranded interference RNA corresponding to the at least one gene selected from the group consisting of FAS, CTLA4, and FOXP3 into the T-lymphocytes.

12. A fragment of a hepatitis C protein which does not suppress activity of dendritic cells and is effective to produce an immunogenic response against hepatitis C infection comprising amino acids 1027-1218 of the NS3 protein or a variant thereof.

13. A method for preparing a therapeutic immunogenic composition for treating hepatitis C infection in a human patient, the composition of claim 1 comprising
providing an amount of isolated autologous dendritic cells;
loading the said dendritic cells with at least one fragment of a hepatitis C protein or a variant thereof, wherein the at least one fragment of a hepatitis C protein comprises amino acids 1027-1218 of the NS3 protein, does not suppress activity of dendritic cells and is effective to produce an immunogenic response against hepatitis C infection and; and
combining said dendritic cells with a pharmaceutically acceptable carrier and/or adjuvant.

14. The method of claim 13, wherein the fragment of a hepatitis C protein is loaded into the dendritic cells by electroporation.

15. The method of claim 13, further comprising introducing into the composition T-lymphocytes isolated from the blood of a patient to be treated, wherein the T-lymphocytes have been activated to produce a Th-1 type response.

16. The method of claim 15, wherein the dendritic cells are further modified to suppress expression of at least one gene selected from the group consisting of CTL4, FAS and FOXP3, and the T-lymphocytes are further modified to suppress expression of at least one gene selected from the group consisting of CTLA4, FAS and FOXP3

17. The method of claim 16, wherein the suppression of the expression of at least one of the genes selected from the group consisting of SOCS-1 and FAS in the dendritic cells and at least one of the genes selected from the group consisting of FAS, CTLA4, and FOXP3 in T-lymphocytes is performed by introducing short double-stranded interference RNA corresponding to the at least one gene selected from the group consisting of SOCS-1 and FAS in the dendritic cells, and FAS, CTLA4, and FOXP3 into the T-lymphocytes respectively.

18. A method for treating, prophylaxis or reducing the severity of hepatitis C virus infection in a patient in need thereof, comprising administering to the patient an effective dose of a composition comprising an amount of isolated autologous dendritic cells loaded with at least one fragment of a hepatitis C protein comprising amino acids 1027-1218 of the NS3 protein, or a variant thereof, wherein the said fragment or the variant thereof does not suppress activity of dendritic cells, and is effective to produce an immunogenic response against hepatitis C infection, and a pharmaceutically acceptable carrier and/or adjuvant.

19. The method of claim 18, further comprising the use of traditional anti hepatitis C virus therapy, after, before or simultaneously with administration of a composition of claim 1.

20. A method for enhancing immunoresponse in a patient suffering from hepatitis C virus infection comprising administering to the patient in need of the treatment the composition of claim 1.

21. A unit dosage form for the treatment or prophylaxis of hepatitis C virus infection in a patient in need thereof, comprising an effective amount of the composition of claim 1.

22. A system for preparing the composition of claim 1 comprising:
at least one fragment of a hepatitis C protein, wherein the fragment of a hepatitis C protein comprises amino acids 1027-1218 of the NS3 protein or a variant thereof, does not suppress activity of dendritic cells and is effective to produce an immunogenic response against hepatitis C infection and allows reduction of the viral load in the patient; and
means for loading the dendritic cells isolated from a patient to be treated with the said fragment of the hepatitis C protein or a variant thereof.

23. The system of claim 22, wherein the means for loading are means for electroporation.
